(19) United States
(12) Patent Application Publication
(10) Pub. No.: US 2011/0091512 A1
(43) Pub. Date: Apr. 21, 2011

(54) HIGHLY POROUS, LARGE POLYMERIC PARTICLES AND METHODS OF PREPARATION AND USE

(75) Inventor: Nai-Hong LI, Edmonton (CA)

(73) Assignee: SUNSTORM RESEARCH CORPORATION, Los Gatos, CA (US)

(21) Appl. No.: 12/676,870

(22) PCT Filed: Sep. 5, 2008
(86) PCT No.: PCT/US2008/075512

§ 371 Date: Jan. 4, 2011

Related U.S. Application Data
(60) Provisional application No. 60/970,261, filed on Sep. 5, 2007.

(51) Int. Cl. C08F 112/08; A61K 9/14

(52) U.S. Cl. 424/401; 521/146; 428/402; 424/489

(57) ABSTRACT

The present invention relates to porous cross-linked polymeric particles having cavities joined by interconnecting pores wherein all or nearly all of the cavities at the interior of each particle communicate with the surface of the particle. The present invention also relates to a process for producing a porous, cross-linked large polymeric particle as well as the product of this process. This process involves combining a continuous phase with an aqueous discontinuous phase to form an emulsion, then placing this emulsion into a mold to produce large particles having shapes derived from a mold, e.g., ellipsoids, spheroids, cylinders, prisms, etc. Also included in the invention are modifications of the particles as well as methods for using the particles in a variety of applications. All described methods, compositions, and articles of manufacture are within the scope of the invention.

### CROSS-REFERENCE TO RELATED APPLICATIONS

This application claims priority to U.S. Provisional Patent Application No. 60/970,261, filed Sep. 5, 2007, which is hereby incorporated by reference in its entirety.

### FIELD OF THE INVENTION

The present invention relates to particles of a cross-linked porous polymeric material and methods for preparing and using such particles. In particular, this invention is directed to a polymeric particle of exceptionally high porosity.

### BACKGROUND OF THE INVENTION

Cross-linked, homogeneous, porous polymeric materials are disclosed in U.S. Pat. No. 4,522,953 (Barby et al., issued Jun. 11, 1985). The described polymeric materials are produced by polymerization of water-in-oil emulsions having a relatively high ratio of water to oil. These emulsions are termed “high internal phase emulsions” and are known in the art as “HIPE” or “HIPE polymers”. HIPE polymers as described in Barby comprise an oil continuous phase including a monomer and a cross-linking agent and an aqueous discontinuous phase. Such emulsions are prepared by subjecting the combined oil and water phases to agitation in the presence of an emulsifier. Polymers are produced from the resultant emulsion by heating. The polymers are then washed to remove undesired components. The disclosed porous polymers have rigid structures containing cavities interconnected by pores in the cavity walls. By choosing appropriate component and process conditions, HIPE polymers with void volumes of 70% or more can be achieved.

Various modifications of HIPE polymers have been described. For instance, U.S. Pat. No. 4,536,521 (Haq, issued Aug. 20, 1985) describes HIPE polymers that can be sulfonated to produce a material that exhibits a high capacity for absorption of ionic solutions. Other functionalized HIPE polymers prepared by a similar process have been described in U.S. Pats. Nos. 4,611,014 (Jomes et al., issued Sep. 9, 1986) and 4,612,334 (Jones et al., Sep. 16, 1986).

Processes for large-scale production of HIPE polymers are known. For instance, U.S. Pat. No. 5,149,720 (DesMarais et al., issued Sep. 22, 1992) discloses a continuous process for preparing HIPE polymers that are suitable for polymerization into absorbent polymers. In addition, a method that facilitates such continuous processes by reducing the curing time of monomers in a HIPE is set forth in U.S. Pat. No. 5,252,619 (Brownscombe et al., issued Oct. 12, 1993). Large-scale production of HIPE polymers by such known processes, however, has been hampered by the lack of a cost-efficient means of removing the unpolymerized emulsion components from the polymers.

Prior art processes for making HIPE polymers produce large blocks of polymeric material the size and shape of the vessel used for polymerization. The problem with producing HIPE polymers in block form is that it is very difficult to wash unpolymerized emulsion components out of a block of low density, highly absorbent material. For many applications, the removal of residual emulsion components is essential. The attempted solution to this problem has been to grind the blocks into particles, but this approach is unsatisfactory because both the drying and milling processes are costly, there is a limit to the size of the particles produced by milling, and fragile HIPE formulations may disintegrate or partially disintegrate during the milling process.

An additional problem with prior art HIPE polymeric blocks is that the blocks have a coating or skin that forms at the interface between the HIPE and the container used for polymerization. (U.S. Pat. No. 4,522,953, Barby et al., issued Jun. 11, 1985, at column 4, lines 1-6). To produce a permeable block, and hence, to produce a useful product, the coating or skin must be removed. Ideally, one would like to be able to produce a polymeric material having the desirable characteristics of HIPE polymers but lacking any coating or skin.

In US patents by Li et al. (U.S. Pat. Nos. 5,583,162; 5,653,922; 5,760,097; 5,863,957; 6,100,306) incorporated herein by reference, HIPE microbeads are described that avoid many of the problems associated with prior art HIPE materials. In particular, these microbeads have a porous, cross-linked, polymeric structure, characterized by cavities joined by interconnecting pores. At least some of the cavities at the interior of each microbead described in these patents communicate with the surface of the particle.

Although the microbeads described by Li et al. have useful applications, there are many other applications that would benefit from materials comprising relatively large high porosity particles of predefined shape, e.g. spheroid, ellipsoid, cylindrical, prismatic, and also exhibit the presence of interconnected cavities.

### SUMMARY OF THE INVENTION

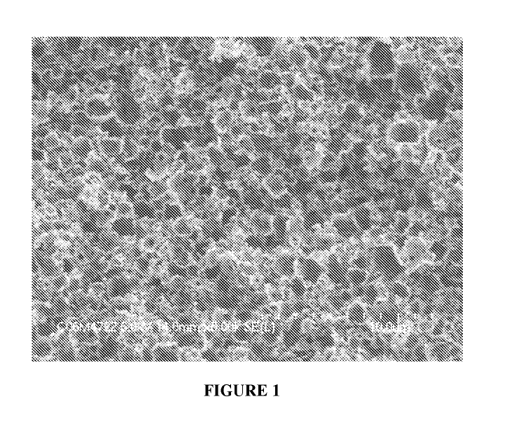

The present invention comprises a process for producing highly porous, cross-linked polymeric particle shapes by mold polymerization that are characterized by cavities joined by interconnecting pores such that the resultant polymers are free from a coating or skin on substantially the entire surface of the particles. Also provided are the particles produced by this process, as well as populations of such particles, alone or in mixtures. The initial steps in this process have been described in the patents by Li et al. The first step is to combine a continuous phase with an aqueous discontinuous phase to form a high internal phase emulsion. The continuous phase of the emulsion comprises a substantially oil-soluble, monofunctional monomer, a substantially oil-soluble, polyfunctional cross-linking agent, and an emulsifier that is suitable for forming a stable water-in-oil emulsion. In the present invention, the emulsion described above is added to a mold form having a predetermined shape (e.g., spheroidal, ellipsoidal, cylindrical, prismoidal, disk, pill or tablet shape). The emulsion contained in the mold is polymerized by any suitable method, e.g. by heating, by photoactivation of a light-sensitive initiator, etc. The resultant material is shown in FIG. 1.

Any suitable polymer precursors that can form the particles of interest can be used. For example, the continuous phase may include monomers and crosslinkers as disclosed by Li et al. (above), e.g. styrene as the monomer, divinylbenzene as the cross-linking agent, and sorbitan monooleate as the emulsifier. In addition, the continuous phase contains an oil-soluble polymerization initiator such as azoisobisbutyronitrile as well as a material such as dodecane, which promotes the formation of interconnecting pores. The aqueous discontinuous phase of at least 70% may include a water-soluble polymerization initiator, e.g. potassium persulfate.

The present invention also encompasses particles that have been modified for use in particular applications. In particular, the present invention includes particles functionalized for absorption of liquids, particles having a gel or pre-gel within the particle cavities and particles having other ingredients or formulations within the particle cavities, as well as processes for producing such particles.

In addition, the present invention includes the use of particles in a variety of applications that would benefit from particles having substantially the entire surface free of coating or skin, including the use of particles as a substrate in separation technologies; the use of the particles in various solid phase synthesis applications; the use of particles as a substrate for immobilizing a molecule such as a polypeptide, an enzyme, an oligonucleotide or other macromolecule; the use of particles in cell culture methods; the use of the particles to contain whole viruses, the use of the particles in gene therapy applications; the use of the particles as carriers of active ingredients such as pharmaceutical agents; the use of particles as carriers for various cosmetic formulations and skin care applications; the use of the particles as a scaffolding for tissue culture applications; the use of the particles as a scaffolding for synthetic cartilage; the use of the particles as a scaffolding for artificial organs, e.g. the liver; the use of the particles to contain various catalysts; the use of the particles for fuel cell applications; the use of the particles as carriers for various adhesives; the use of the particles as a low-density filler; and the use of the particles in conjunction with conductive polymer applications.

### DETAILED DESCRIPTION OF THE INVENTION

### The Particle

The present invention includes a cross-linked porous polymeric material, termed “particles,” wherein the particles were formed by transferring a stabilized high internal phase emulsion into a mold having a predetermined shape, and polymerizing the HIPE to form particles with a shape predetermined by the mold. The present invention also includes a process for making such a material. A particle is typically produced by first filling a mold form having a spheroid, ellipsoid, cylindrical or the like shape with a high internal phase emulsion, termed a “HIPE”. The particle of the present invention thus has many of the desirable physical characteristics of prior art HIPE polymers (such as those disclosed in U.S. Pat. No. 4,522,953, Barby et al., issued Jun. 11, 1985, which is incorporated by reference herein in its entirety) and the patents of Li et al. as described above and incorporated by reference herein in its entirety. Specifically, the particle has a very low density due to the presence of cavities joined by interconnecting pores. The void volume of the particle is at least about 70% and, in a preferred embodiment, is at least about 90%. The measured dry density, determined from the volume of the mold and the weight of the particle, is less than about 0.20 gm/cm³, and typically less than about 0.10 gm/cm³. This high porosity and low density gives the particle exceptional absorbency. Furthermore, because the interconnectedness of the cavities in the particle allows liquids to flow through the particle, the particle provides an excellent substrate for use in biotechnology and biomedical applications such as, for example, chromatographic separation of biomolecules and biomolecule synthesis, gene therapy applications or as scaffolding for tissue engineering applications.

The average particle size typically ranges from a volume of about 65 mm³ to a volume of about 525 mm³, depending on the size of the mold form used. In some embodiments, the particles are preferably in the volume range about 100 mm³ to about 300 mm³. Use of particles in the volume range of about 65 mm³ to about 525 mm³ facilitates efficient washing of the particle to remove residual unpolymerized emulsion components. Also, since mold forms are utilized, the process of the present invention can be used to produce particles of a substantially uniform size and shape. This allows the wash conditions to be optimized to ensure that each particle in a batch has been thoroughly washed, and allows for consistency between batches. Thus, the particles of this invention, unlike prior art HIPE blocks, can be washed with relative ease. Furthermore, since a mold form is used, yield of the particular size determined by the mold form is usually very high, and may approach 100%.

An additional feature of the particle of the present invention is that the particle is highly “skinless” such that nearly all interior cavities and pores communicate with the surface of the particle and substantially the entire particle surface is free from a coating or skin. This feature contributes to improvements in washing of particles described in prior art since there is substantially no coating or skin present on the surface. Washing solvents can easily flow through the entire volume of the particles when there is substantially no coating or skin on the particle surface. In some embodiments, at least 50%, at least 60%, at least 70%, at least 80%, at least 85%, at least 90%, or at least 95% of the cavities at the interior of the particle communicate with the surface of the particle. This feature of the present invention facilitates cost-efficient scale-up of HIPE polymer production.

Also provided is a method of forming such a particle, comprising preparing a HIPE, transferring the HIPE to a mold having one or more predetermined shapes able to produce a particle of the size and volume of interest as described herein, and polymerizing the HIPE to form such particles.

Further provided is a population comprising particles of substantially uniform shape. By substantially uniform shape is meant a shape which varies on average by less than 20% in each dimension, preferably less than 10%, and desirably about 5% or less. The population may comprise more than one subpopulations each having a substantially uniform shape differing from the other. Additionally or alternatively, different subpopulations may differ in color, which may or may not reflect the identity of different included compositions within different particle subpopulations.

### Skin of the Particle

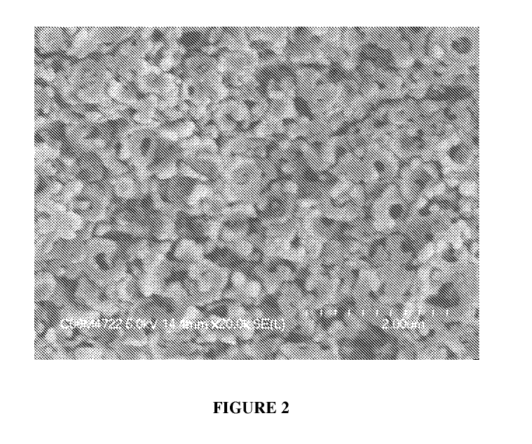

Shapes formed by prior art methods such as described by Barby (U.S. Pat. No. 4,522,953) yield a “skin” at the interface between the particle and the mold surface. Surprisingly, the method described in this invention does not yield a skin on the particles. Internal structure of large particles of this invention is shown in FIG. 1 and the external surface of these particles is illustrated in FIG. 2. The similarity of the internal structure and external surface structure is apparent. Without wishing to be bound by theory, possible contributing factors in producing relatively “skinless” particles during mold polymerization may result from the use of molds of relatively small volume (below about 525 mm³), occasional use of organic solvents in the formulation, selection of appropriate mold compositions that resist skin formation, possibly selection of appropriate curing temperature profiles, or combinations of some or all of these.

Thus, the particles and compositions of this invention offer advantages in applications that benefit from utilizing particles that have substantially no coating or skin on the surface. This feature renders the particle immediately useful as an absorbent material and also as a solid support in a variety of chemical, biotechnology, biomedical and related applications, including chromatographic separations, solid phase synthesis, immobilization of antibodies or enzymes, cell culture and tissue engineering. These particles are also useful in consumer applications such as cosmetics, feminine care, oral care and wound treatment. Moreover, many of the physical characteristics of the particle, such as void volume and cavity size, are controllable. Therefore, different types of particles, specialized for different uses, can be produced. A description of the general process for producing the particle according to this invention is presented below.

### DEFINITIONS

Before the present invention is further described, it is to be understood that this invention is not limited to the particular methodology, devices, solutions or apparatuses described, as such methods, devices, solutions or apparatuses can, of course, vary. It is also to be understood that the terminology used herein is for the purpose of describing particular embodiments only, and is not intended to limit the scope of the present invention.

Use of the singular forms “a,” “an,” and “the” include plural references unless the context clearly dictates otherwise. Thus, for example, reference to “a monomer” includes a plurality of monomers, reference to “a particle” includes a plurality of such particles, reference to “a cosmetic” includes a plurality of cosmetics, and the like. Terms such as “connected,” “attached,” “linked,” and the like are used interchangeably herein and encompass direct as well as indirect connection, attachment, or linkage unless the context clearly dictates otherwise, and includes chemical couplings as well as nonchemical binding or other association. Thus, these terms intend that the particles, chemicals, labels, etc., which are “linked” may be physically linked by, for example, covalent chemical bonds, physical forces such van der Waals or hydrophobic interactions, encapsulation, embedding, or the like.

Where a range of values is recited, it is to be understood that each intervening integer value, and each fraction thereof, between the recited upper and lower limits of that range is also specifically disclosed. The upper and lower limits of any range can independently be included in or excluded from the range, and each range where either, neither or both limits are included is also encompassed within the invention. Where a value being discussed has inherent limits, for example where a component can be present at a concentration of from 0 to 100%, or where the pH of an aqueous solution can range from 1 to 14, those inherent limits are specifically disclosed. Where a value is explicitly recited, it is to be understood that values which are about the same quantity or amount as the recited value are also within the scope of the invention. Where a combination is disclosed, each subcombination of the elements of that combination is also specifically disclosed and is within the scope of the invention. For any element of an invention for which a plurality of options are disclosed, examples of that invention in which each of those options is individually excluded along with all possible combinations of excluded options are hereby disclosed.

Unless defined otherwise or the context clearly dictates otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

All publications mentioned herein are hereby incorporated by reference for the purpose of disclosing and describing the particular materials and methodologies for which the reference was cited. The publications discussed herein are provided solely for their disclosure prior to the filing date of the present application. Nothing herein is to be construed as an admission that the invention is not entitled to antedate such disclosure by virtue of prior invention.

The term “particles” refers to a cross-linked porous polymeric material produced by transferring a stabilized high internal phase emulsion into a mold and allowing this HIPE to polymerize to yield a predetermined shape reflecting the shape of the mold (e.g., spheroid, ellipsoid, cylindrical, geometric prisms, etc.).

As applied to the components of a HIPE, the phrase “substantially oil-soluble” indicates that any component present in the aqueous phase is present at such a low concentration that polymerization of aqueous monomer is less than about 5 weight percent of polymerizable monomer.

The term “void volume” refers to the volume of a particle that does not comprise polymeric material. In other words, the void volume of a particle comprises the total volume of the cavities. Void volume is expressed as a percentage of the total particle volume. The total volume of the particles is determined by the mold size. The weight of the particles is measured directly. The density of the polymer is known, and can alternately be measured by polymerizing the oil phase in an identical mold without an aqueous discontinuous phase to produce a solid particle having a measurable weight and volume. The unoccupied (void) volume of the porous particles can thus be calculated directly from the weight, density and volume.

As used herein, the term “cavity size” refers to the average diameter of the cavities present in a particle.

As used herein, the term “porogen” refers to an organic compound that, when included in the continuous phase of a HIPE, promotes the formation of pores connecting the cavities in a particle. Exemplary porogens include dodecane, toluene, cyclohexanol, n-heptane, isooctane, and petroleum ether. The porogen is typically present in the continuous phase at a concentration in the range of about 10 to about 60 weight percent.

The abbreviation “DVB” refers to “divinylbenzene”; the abbreviation “AIBN” refers to “azoisobisbutyronitrile”; and the abbreviation “PVA” refers to “poly(vinylalcohol)”, which is typically produced by hydrolysis of poly(vinylacetate).

### Production of the Particle

The particles of the present invention are conveniently produced from a HIPE, which comprises an emulsion of an aqueous discontinuous phase in an oil continuous phase. Once formed, the HIPE is added to a mold form. Polymerization then converts the liquid HIPE to solid particles.

### Components of the High Internal Phase Emulsion

The relative amounts of the two HIPE phases are, among other parameters, important determinants of the physical properties of the particle. In particular, the percentage of the aqueous discontinuous phase affects void volume, density, and cavity size. For the emulsions used to produce preferred particles, the percentage of aqueous discontinuous phase is generally in the range of about 70% to about 98%, more preferably at least 75%, and most preferably at least 80%. Desirable particles for cosmetic applications are produced at approximately 95% porosity (range of 90-99% porosity), as the particle apparently disappears when crushed to distribute the composition.

The continuous phase of the emulsion comprises a monomer, a cross-linking agent, and an emulsifier that is suitable for forming a stable water-in-oil emulsion. Any suitable monomer component(s) can be used; for example, those used in prior art HIPE polymers, and can be any substantially oil-soluble, monofunctional monomer. Of particular interest are vinyl or derivatized vinyl, derivatized with functional groups such as alkyl, aryl, acids, bases, esters, halogens, ethers, alcohols, and combinations of functional groups; suitable monomers are commercially available. In some embodiments, the monomer type is a styrene-based monomer, such as styrene, 4-methylstyrene, 4-ethylstyrene, chloromethyl styrene, 4-t-BOC-hydroxystyrene. The monomer component can be a single monomer type or a mixture of types. The monomer component is typically present in a concentration of about 5% to about 90% by weight of the continuous phase. The concentration of the monomer component is preferably about 15% to about 50% of the continuous phase, more preferably, about 16% to about 38%.

The cross-linking agent can be selected from a wide variety of substantially oil-soluble, polyfunctional crosslinkers. Suitable cross-linking agents are known in the art and include divinyl aromatic compounds, such as divinylbenzene (DVB). Other types of cross-linking agents, such as di- or triacrylic compounds and triallyl isocyanurate, can also be employed. The cross-linking agent can be a single cross-linker type or a mixture of types. The cross-linking agent is generally present in a concentration of about 1% to about 90% by weight of the continuous phase. Preferably, the concentration of the cross-linking agent is less than about 35%, and more preferably is less than about 30%. In some embodiments, the cross-linking agent is in the range of about 15% to about 50% of the continuous phase, more preferably, about 16% to about 38%. In some embodiments, the cross-linking agent is present at a concentration of about 16 to about 25%, and may be about 20%, or in the range of about 1 to about 20%.

In addition to a monomer and a cross-linking agent, the continuous phase comprises an oil-soluble emulsifier that promotes the formation of a stable emulsion. The emulsifier can be any nonionic, cationic, anionic, or amphoteric emulsifier or combination of emulsifiers that promotes the formation of a stable emulsion. Suitable emulsifiers are known in the art and include sorbitan fatty acid esters, polyglycerol fatty acid esters, and polyoxyethylene fatty acids and esters. In some embodiments, the emulsifier is sorbitan monooleate (sold as SPAN 80). The emulsifier is generally present at a concentration of about 3% to about 50% by weight of the continuous phase. Preferably, the concentration of the emulsifier is about 10% to about 25% of the continuous phase. More preferably, the concentration is about 15% to about 20%.

In some embodiments, the continuous phase also contains an oil-soluble polymerization initiator and a porogen. The initiator can be any oil-soluble initiator that permits the formation of a stable emulsion, such as an azo initiator or a peroxide initiator. A preferred initiator is azoisobisbutyronitrile (AIBN). In some embodiments, the initiator is selected from the group consisting of AIBN, benzoyl peroxide, lauroyl peroxide, and a VAZO initiator. The initiator can be present in a concentration of up to about 5 weight percent of total polymerizable monomer (monomer component plus cross-linking agent) in the continuous phase. The concentration of the initiator is preferably about 0.5 to about 1.5 weight percent of total polymerizable monomer, more preferably, about 1.2 weight percent.

The porogen of the present invention can be any organic compound or combination of compounds that permits the formation of a stable emulsion while promoting pore formation without becoming incoporated into the polymer, provided that the compound is a good solvent for the monomers employed, and preferably is a poor solvent for the polymer produced. Suitable porogens include dodecane, toluene, cyclohexanol, n-heptane, isooctane, and petroleum ether. A preferred porogen is dodecane. The porogen is generally present in a concentration of about 10 to about 60 weight percent of the continuous phase. The porogen concentration affects the size and number of pores connecting the cavities in the particle. Specifically, increasing the porogen concentration increases the size and number of interconnecting pores; while decreasing the porogen concentration decreases the size and number of pores. Preferably, the porogen concentration is about 25 to about 40 weight percent of the continuous phase. More preferably, the concentration is about 30 to about 35 weight percent.

In some embodiments, the aqueous discontinuous phase of a HIPE comprises a water-soluble polymerization initiator. In these cases, the initiator can be any suitable water-soluble initiator. Such initiators are known and include peroxide compounds such as sodium, potassium, and ammonium persulfates; sodium peracetate; sodium percarbonate and the like. A preferred initiator is potassium persulfate. The initiator can be present in a concentration of up to about 5 weight percent of the aqueous discontinuous phase. Preferably, the concentration of the initiator is about 0.5 to about 2 weight percent of the aqueous discontinuous phase.

### Cavities and Pores

The cavities reflect the included aqueous discontinuous phase present during polymerization. Due to surface tension effects, the included aqueous phase droplets form a generally spherical shape, reflected in the cavities present in the resulting polymer. In some embodiments, the adjacent cavities are interconnected by a plurality of pores of smaller size than the cavities; the pores form generally circular connections between cavities, and have been observed to form one or more subpopulations of pores of generally similar sizes. In some embodiments, the cavities comprise six interconnecting pores. In some embodiments, the average interconnecting pore diameter is at least 0.5 microns. In some embodiments, the average interconnecting pore diameter is 20% or less than the average cavity diameter. In some embodiments, the ratio of cavity diameter to pore diameter is about 7:1.

### Production of a High Internal Phase Emulsion

The first step in the production of a HIPE-based particle is the formation of a high internal phase emulsion. A HIPE can be prepared by any of the prior art methods, for example as disclosed in U.S. Pat. No. 4,522,953 (Barby et al., issued Jun. 11, 1985). Briefly, a HIPE is formed by combining the continuous and aqueous discontinuous phases while subjecting the combination to shear agitation. Generally, a mixing or agitation device such as a pin impeller is used. For cosmetic uses, generally a porosity of at least about 90% or more is desired to permit minimal residual polymer after the cosmetic is applied.

The extent and duration of shear agitation must be sufficient to form a stable emulsion. As shear agitation is inversely related to cavity size, the agitation can be increased or decreased to obtain a particle with smaller or larger cavities, respectively. In some embodiments, a HIPE is prepared using a Gifford-Wood Homogenizer-Mixer (Model 1-LV), set at 1400 rpm. At this mixing speed, the HIPE is produced in approximately 5 minutes. In another embodiment, a HIPE is prepared using an air-powered version of the above mixer (Model 1-LAV), with air pressure set at 5-10 psi for approximately 5-10 minutes. The HIPE can be formed in a batchwise or a continuous process, such as that disclosed in U.S. Pat. No. 5,149,720 (DesMarais et al., issued Sep. 22, 1992).

As particles of certain desired sizes could not be consistently formed from emulsion suspension and polymerization, mold formation of particles is preferred for larger size (greater than four, five or six mm in smallest diameter).

It is also possible to produce polymers having structures more resistant to crushing, having less than 96% porosity (e.g., app. 90% porosity). In such embodiments, the shards of broken polymer produced on crushing can produce an exfoliating effect as the formulation is applied to the skin.

### Addition of a HIPE to a Mold Form

Once formed, the HIPE can be added to a mold form through any suitable technique, for example using a transfer apparatus such as a syringe, or by carefully pouring the emulsion into the mold cavities.

The mold can have one or more predetermined shapes for forming particles of the desired shape and/or size. In some embodiments, the molds produce particles of a size resulting in single particles having a useful distribution amount of cosmetic ingredient (for example, one particle would cover a part of the body of interest for the particular cosmetic).

In some embodiments, particles may be made of one or more shapes, one or more colors (by including a colorant which can be incorporated into the particles and/or the included composition(s)), or both. Mixtures of particles may be provided. In some embodiments, the colorant may be used to provide a color indicator of the composition incorporated in individual particles.

### Polymerization of HIPE Particles

Once the HIPE has been added to the mold form, the HIPE can be polymerized by any suitable technique (e.g., by heating, irradiation). For example, to initiate polymerization by heating, the temperature of the filled mold is increased above ambient temperature to initiate polymerization. Polymerization conditions vary depending upon the composition of the HIPE. For example, the monomer or monomer mixture and the polymerization initiator(s) are particularly important determinants of polymerization temperature. Furthermore, the conditions must be selected such that a stable HIPE can be produced during the time necessary for polymerization. The determination of a suitable polymerization temperature for a given HIPE is within the level of skill in the art. In general, the temperature should not be elevated above 85° C. because high temperatures can cause the emulsion to break. In one example, when AIBN is the oil-soluble initiator and potassium persulfate is the water-soluble initiator, styrene monomers are polymerized by maintaining the mold at 60° C. overnight (approximately 18 hours).

Removal of the Particle from the Mold

After polymerization, the particles can be removed by any suitable method, typically by turning the mold over and tapping or shaking the mold to dislodge the particles. After polymerization, the particles are generally stable to reasonable manipulation, but are susceptible to crushing forces as can be manually applied by intentional fingertip or hand pressure, for example to distribute a cosmetic formulation loaded into the cavities within the particles.

### Washing of the Particle

The polymerization step converts the HIPE to shaped particles. As discussed above, these particles are generally washed to remove any undesired components of the HIPE. The particle can be washed with any liquid that can solubilize such components without affecting the stability of the particle. More than one cycle of washing may be required. In one washing regimen, the particle is washed five times with water, followed by acetone extraction for roughly a day in a Soxhlet extractor. The particle can then be dried through any suitable technique; a number of methods are known in the art. In some embodiments, the particle is air-dried for two days or is dried under vacuum at 50° C. overnight.

Loading Substances into the Particle

The utility of the particle can be increased by loading a gel or other formulated material to the particle interior according to the methods described in U.S. Pat. No. 4,965,289 (Sherrington, issued Oct. 23, 1990). The gel can be formed in or added to the particle cavities and may be linked to the particle surface. The gel may bear either acidic or basic groups, depending on whether the particle substrate is to serve as an anion-exchange resin or a cation-exchange resin, respectively.

Other substances can be loaded into the particle according to intended applications. For example, various cosmetics or skin care formulations can be added to the particles. Particles prepared according to this invention are amenable to incorporation of gels or other substances due to the surface porosity. Exemplary cosmetics suitable for loading into the particles include those sold by Johnson and Johnson, Pierre Fabre, Chanel, Este Lauder, and others.

### Modifications of the Particle

The particle can be used for a variety of applications, notably, as an absorbent material, as a solid support in biotechnology applications and as a carrier of active ingredients or other formulated compounds. A particle-based absorbent can be used, for example, to transport solvents, to absorb body fluids, and as an adhesive microcarrier. Biotechnology applications include chromatographic separations, solid phase synthesis, immobilization of antibodies or enzymes, gene therapy applications, and microbial and mammalian cell culture as well as tissue engineering. The basic particle can be modified in a variety of ways to produce particles that are specialized for particular applications.

Functionalized particles can be produced by known methods, disclosed, for example, in U.S. Pat. No. 4,611,014 (Jomes et al., issued Sep. 9, 1986). Briefly, the functionalized particle is generally prepared indirectly by chemical modification of a preformed particle bearing a reactive group such as bromo or chloromethyl.

A particle suitable for subsequent chemical modification can be prepared by polymerization of monomers such as chloromethyl styrene or 4-t-BOC-hydroxystyrene. Other suitable monomers are styrene, α-methyl styrene, or other substituted styrene or vinyl aromatic monomers that, after polymerization, can be chloromethylated to produce a reactive particle intermediate that can be subsequently converted to a functionalized particle.

Monomers that do not bear reactive groups (including the cross-linking agent) can be incorporated into the particle at levels up to about 20% or more. To produce HIPE-based particles, however, such monomers must permit the formation of a stable HIPE. The concentration of the reactive monomer should generally be sufficiently high to ensure that the functionalized particle generated after chemical modification bears ionic or polar functional groups on a minimum of about 30% of the monomer residues.

Chemical modification of the reactive particle intermediate is carried out by a variety of conventional methods. Preferred exemplary methods for producing amine-, amine salt-, and cationic quaternary ammonium-functionalized particles are described in detail in Examples 2 to 4, respectively.

In another embodiment, particles bearing ionic or polar groups can be prepared directly by emulsification and polymerization of an appropriate substantially oil-soluble monomer.

Production of a Stable Carbon Structure from the Particle

A particle can be converted to a porous carboniferous material that retains the original structure of particle cavities and interconnecting pores. This material is useful, for example, as a sorption or filtration medium and as a solid support in a variety of biotechnology applications (described further in the next section). In addition, the carboniferous particle can be used as an electrode material in batteries and super-capacitors. Battery electrode materials preferably have large lattice spacing, such as that of the particle. Large lattice spacing reduces or eliminates lattice expansion and contraction during battery operation, extending battery cycle lifetimes. Super-capacitors require highly conductive electrodes. The particle is ideally suited for this application because the interconnectedness of the particle renders it highly conductive.

To produce a carboniferous particle, a stable particle is heated in an inert atmosphere as disclosed for HIPE polymers in U.S. Pat. No. 4,775,655 (Edwards et al., issued Oct. 4, 1988). The ability of the particle to withstand this heat treatment varies depending on the monomer or monomers used. Some monomers, such as styrene-based monomers, yield particles that must be stabilized against depolymerization during heating.

The modification required to stabilize such particles can take many forms. Particle components and process conditions can be selected to achieve a high level of cross-linking or to include chemical entities that reduce or prevent depolymerization under the heating conditions employed. Suitable stabilizing chemical entities include the halogens; sulfonates; and chloromethyl, methoxy, nitro, and cyano groups. For maximum thermal stability, the level of cross-linking is preferably greater than about 20% and the degree of any other chemical modification is at least about 50%. Stabilizing entities can be introduced into the particle after its formation or by selection of appropriately modified monomers.

Once stabilized, the particle is heated in an inert atmosphere to a temperature of at least about 500° C. In some cases, to remove undesired components, the temperature may be raised to at least about 1200° C.

### Use of the Particle in Cell Culture and Tissue Engineering

In addition to the above applications, the particle is also useful in cell culture. High density cell culture generally requires that cells be fed by continuous perfusion with growth medium. Suspension cultures satisfy this requirement; however, shear effects limit aeration at high cell concentration. The particle protects cells from these shear effects and can be used in conventional stirred or airlift bioreactors.

To prepare a particle for use in culturing eukaryotic or prokaryotic cells, the particle is generally sterilized by any of the many well-known sterilization methods. Suitable methods include irradiation, ethylene oxide treatment, and, preferably, autoclaving. Sterile particles are then placed in a culture vessel with the growth medium suitable for the cells to be cultured. Suitable growth media are known for suspension cultures. An inoculum of cells is added and the culture is maintained under conditions suitable for cell attachment to the particles. The culture volume is then generally increased, and the culture is maintained in the same manner as prior art suspension cultures.

Particles can be used in cell culture or tissue engineering without modification; however, the particles can also be modified to improve cell attachment, growth, and the production of specific proteins. For instance, a variety of bridging molecules can be used to attach cells to the particles. Suitable bridging molecules include antibodies, lectins, glutaraldehyde, polycationic species (e.g., poly-L-lysine), and/or matrix or basement membrane molecules (fibronectin, vitronectin, thrombospondin, collagen, etc.). In addition, sulfonation of particles can increase cell attachment rates in some instances. Inoculating particles with cells for cell culture or tissue engineering is greatly enhanced using particles prepared according to this invention since substantially all the surface is porous and available for loading or inoculation.

### Use of the Particle in Drug Delivery Applications

Pharmaceutically active ingredients can be loaded into these large particles. The larger HIPE shapes of this invention could be used in a similar fashion, but could be used as a single, monolithic structure that could be taken orally. In some cases, these monoliths could be implanted in patients to provide drug release over extended periods.

This invention is further illustrated by the following specific but non-limiting examples. Procedures that are constructively reduced to practice are described in the present tense, and procedures that have been carried out in the laboratory are set forth in the past tense.

### Example 1

### Production of Particles

Exemplary preferred large particles were prepared according to the following protocol:

Materials:
(1) monomers: styrene—(Aldrich)
(2) cross-linker: divinylbenzene(DVB)—(Aldrich, 55% purity)
(3) initiator: AIBN
(4) porogen solvent: Dodecane—(Aldrich)
(5) surfactant: Span 80—(Aldrich)
1. Prepare a continuous phase by combining 24 gm styrene-based monomer, 28 gm DVB, 8.00 gm span 80, 0.8 gm AIBN; and 35.5 gm dodecane with stirring at room temperature.
2. Prepare an aqueous discontinuous phase by adding 0.78 gm potassium persulfate to 840 ml of distilled water. Stir the continuous phase at approximately 1400 rpm, and then add the aqueous discontinuous phase to the continuous phase at a flow rate of 20 ml/minute. Stir the combined phases at 1400 rpm for approximately 5-10 minutes to form a stable HIPE.
3. Polymerization: The emulsion prepared according to above table was transferred into a tray containing cavities of a predetermined size and shape (NUNC™ TC MICROWELL 96U). A 100 mL syringe is filled with the HIPE and injected in the desired tray cavities in a sufficient amount for the desired final volume. The tray was then sealed and placed into an oven at a temperature of 50-55 degrees C. for 4 hrs and then increased to 60-65 degrees C. overnight.
4. After polymerization and curing, the particles were washed with water, methanol and acetone to completely remove the un-polymerized monomer, initiator residue and surfactant. The particles were allowed to air-dry overnight. This yielded particles of approximately 8 mm in length and 3 mm in width, having porosity of approximately 90%.

### Example 2

### Functionalization of Particles for Absorption of Acids Using Amine Groups

Diethylamine-functionalized particles are produced from chloromethyl styrene particles as described in Example 1. The particles are air-dried overnight and Soxhlet extracted for 15 hours with 200 ml hexane to remove residual unpolymerized components. 5 gm of particles are then refluxed with 150 ml aqueous diethylamine for 20 hours. The resultant diethylamine-functionalized particles are 85% substituted and have a capacity of 1.5 mM/gm. 1 gm of this material absorbs 20 ml of 1N sulfuric acid.

### Example 3

### Functionalization of Particles for Absorption of Acids Using Amine Salts

To produce a dihexylammonium salt, dihexylamine-functionalized particles are prepared as described above in Example 3 for diethylamine-functionalized particles. 1 gm dihexylamine-functionalized particles are then added to 100 ml methanolic HCl and stirred for 30 minutes. The counterion of the resultant salt is chloride. The dihexylammonium chloride-functionalized particles are collected by filtration, washed with 3 times with 50 ml methanol, and air-dried overnight. The resultant particles are 70% substituted.

### Example 4

### Functionalization of Particles for Absorption of Acids Using Quaternary Ammonium Groups

To produce a dimethyldecylammonium salt, chloromethylstyrene particles are prepared as described in Example 1. The particles are air-dried overnight and Soxhlet extracted with hexane to remove residual unpolymerized components. 1 gm particles are then filled under vacuum with a 10-fold molar excess of ethanolic amine and refluxed for 7 hours. The counterion of the resultant salt is chloride. The dimethyldecylammonium chloride-functionalized particles are collected by filtration, washed twice with 50 ml ethanol and twice with 50 ml methanol, and then air-dried overnight. The resultant particles are 70% substituted.

### Example 5

### Functionalization of Particles for Absorption of Aqueous Solutions Using Amine Salts

To produce a dimethylammonium salt, diethylamine-functionalized particles are prepared as described in Example 3. The particles are air-dried overnight and Soxhlet extracted with hexane to remove residual unpolymerized components. 1 gm particles are then added to 100 ml methanolic HCl and stirred for 30 minutes. The counterion of the resultant salt is chloride. The diethylamine chloride-functionalized particles are 85% substituted.

### Example 6

### Functionalization of Particles for Absorption of Aqueous Solutions Using Quaternary Ammonium Groups

To produce a dimethyldecylammonium salt, chloromethylstyrene particles prepared as described in Example 1. The particles are air-dried overnight and Soxhlet extracted with hexane to remove residual unpolymerized components. 1 gm particles are then treated with 100 ml aqueous amine for 30 minutes. The resultant dimethyldecylammonium chloride-functionalized particles are 85% substituted.

### Example 7

### Functionalization of Particles for Absorption of Aqueous Solutions Using Alkoxylate Groups

Ethoxylated particles are prepared from chloromethylstyrene particles prepared as described in Example 1. The particles are air-dried overnight and Soxhlet extracted with hexane to remove residual unpolymerized components. 1 gm particles are then treated with 100 ml of an anionic form of a polyethylene glycol (PEG) containing 8-9 ethylene glycol monomers in excess PEG as solvent. The reactants are heated at 95 degrees C. for 2 hours. The resultant ethoxylated particles are 90% substituted.

### Example 8

### Functionalization of Particles for Absorption of Aqueous Solutions Using Sulfonate Groups

Sulfonate-functionalized particles are produced from styrene particles prepared as described in Example 1. The particles are dried under vacuum at 50 degrees C. for two days. 10 gm of particles were then added to a 500 ml flask containing a mixture of 200 ml of chloroform and 50 ml of chlorosulphonic acid. The flask is shaken at room temperature for two days. The sulphonate-functionalized particles are collected by filtration and washed sequentially with 250 ml each of chloroform, methylene chloride, acetone, and methanol. The particles are soaked in 300 ml 10% aqueous sodium hydroxide overnight and then washed with water until the eluate reaches neutral pH. The density of the resultant material is 0.067 gm/ml of dried particles and the capacity is 2.5 mM/gm. 1 gm of this material absorbs 23.5 gm of water.

### Example 9

### Production of Stable Carbon Structures from Chloromethylstyrene Particles

3-chloromethylstyrene particles are prepared as described in Example 1 such that the level of crosslinking is between 20-40% and the void volume is 90%. 1 gm particles are then placed in an electrically heated tube furnace, and the temperature is increased to 600 degrees C. in an oxygen-free nitrogen atmosphere. The rate of heating is generally maintained below 5 degrees C. per minute, and in the range of 180 degrees C. to 380 degrees C., the rate of heating does not exceed 2 degrees C. per minute. After the heating process, the particles are cooled to ambient temperature in an inert atmosphere to prevent oxidation by air.

### Example 10

### Production of Gel-Filled Particles for Use as a Substrate for Protein Synthesis

Particles with a void volume of 90%, a density of 0.047 gm/cm, an average cavity diameter in the range of 1-50 um, and which are 10% crosslinked are prepared as described in Example 1. The gel employed is poly(N-(2-(4-acetoxyphenyl)ethyl)-acrylamide). To produce a solution of gel precursors, 2.5 gm of monomer, 0.075 gm of the crosslinking agent ethylene bis(acrylamide), and 0.1 gm of the initiator AIBN is added to 10 ml of the swelling agent dichloroethane. The gel precursor solution is then deoxygenated by purging with nitrogen.

0.7 gm of particles is added to the gel precursor solution and polymerization is initiated by heating the mixture at 60 degrees C. while rotating the sample on a rotary evaporator modified for reflux. The dichloroethane swells the particles, allowing the gel precursors to penetrate the particle and form a polyamide that becomes interpenetrated with the polymer chains of the particle. After 1 hour, the gel-filled particles (hereinafter “composite”) are washed with 50 ml dimethyl formamide (DMF) and 50 ml diethyl ether and then vacuum dried. The yield of composite is 2.7 gm.

To produce chemical groups within the composite, 0.25 gm of the composite is treated with 50 ml of a 5% solution of hydrazine hydrate in DMF for 5 minutes. This treatment provides free phenolic functionalities within the gel matrix that act as chemical groups for peptide synthesis.

### Example 11

### Use of Particles in High Density Cell Culture

To produce particles suitable for mammalian cell culture, sulfonated particles are prepared as described in Example 8 and are then wetted in a 70% ethanol solution and autoclaved at 121 degrees C. for 15 minutes. The particles are then washed twice with sterile phosphate-buffered saline and once with complete growth medium. 500 mg of the sterile particles are placed in a 500 ml roller bottle that has been siliconized to prevent attachment of the cells to the bottle.

An inoculum of 5×10⁷ baby hamster kidney cells in 50 ml of growth medium (containing 10% fetal calf serum) is added to the roller bottle. The inoculum is incubated with the particles for 8 hours at 37 degrees C. with periodic agitation to allow cell attachment to the particles. The culture volume is then increased to 100 ml, and the roller bottle is gassed with an air-CO² (95:5) mixture and placed in a roller apparatus. Growth medium is replaced whenever the glucose concentration drops below 1 gm/liter.

### Example 12

### Use of Particles as Carriers for Cosmetic Formulations

Large particles prepared according to Example 1 are washed and dried as necessary. The volume of these particles is calculated based upon their geometric shape and dimensions. The available porosity is determined by the formulation selected in the procedure of Example 1. A quantity of liquid ingredients such as used in cosmetic formulations is selected to slightly underfill the cavities within the particles. This quantity of ingredients is then added to a reservoir containing the particles. The liquid formulation is absorbed by capillary forces and fills the particles. Some slight agitation may be required to achieve complete filling. The resulting particles may be picked up without release of ingredients due to the strong capillary forces acting on the composite. Release of the cosmetic formulation can be achieved by crushing the composite particle.

### Example 13

### Production and Loading of Particles of Different Composition

The method of claim 1 was performed using the following variations:

Materials:
(1) monomers: styrene—from Aldrich
(2) cross-linker: divinylbenzene(DVB)—from Aldrich, 55% purity
(3) initiator: AIBN
(4) porogen solvent: Dodecane—from Aldrich
(5) surfactant: Span 80—from Aldrich

### Preparation of High Internal Phase Emulsion

% of HIPE

| Ingredient | 82% | 90% | 92% | 95% |
|---|---|---|---|---|
| Styrene (g) | 23.4 | 24 | 12.1 | 13.3 |
| DVB (g) | 28.0 | 28 | 13.9 | 15.3 |
| AIBN (g) | 0.8 | 0.8 | 1.0 | 1.0 |
| porogen (g) | 32.0 | 30.5 | 15.2 | 15 |
| Water (ml) | 400 | 840 | 530 | 800 |

The emulsion prepared according to above table was transferred into model tray containing cavities of appropriate size. The tray was sealed and placed into an oven with temperature of 50-55 degrees C. for 4 hrs and then at 60-65 degrees C. overnight. After polymerization and curing, the particles were washed with water, methanol and acetone to completely remove the un-polymerized monomer, residue of initiator and surfactant.

### Characterization of Various Particles Having Different Porosity

Particles with % of porosity

| | 82% | 90% | 92% | 95% |
|---|---|---|---|---|
| Weight per particle | 0.0732 | 0.0208 | 0.0148 | 0.0058 |
| (g) | | | | |

The particles were found to have loading capacities of approximately 5-7 grams per gram or particle. Loading times varied from a few minutes to more than an hour, depending on the viscosity of the composition being loaded.

What is claimed is:

1-38. (canceled)

39. A molded porous crosslinked polymeric particle having spherical cavities joined by interconnecting pores, wherein the particle has a void volume of at least 70% and retains the shape of the mold in which it was polymerized.

40. The particle of claim 39, wherein the particle is washed of residual material without first grinding the particle into smaller pieces.

41. The particle of claim 39, wherein the particle has a void volume of at least 75%, at least 80%, at least 85%, at least 90%, at least 95%, or at least 97%.

42. The particle of claim 39, wherein the particle has a measured density of less than about 0.20 gm/cm³ or less than about 0.10 gm/cm³.

43. The particle of any claim 39, wherein at least 50%, at least 60%, at least 70%, at least 80%, at least 85%, at least 90%, or at least 95% of the cavities at the interior of the particle communicate with the surface of the particle.

44. The particle of claim 39, wherein the particle has a volume of from about 65 mm³ to about 525 mm³.

45. The particle of claim 39, wherein the particle has a spheroidal, ellipsoidal, cylindrical, prismoidal, disk, pill or tablet shape.

46. The particle of claim 39, wherein the cavities comprise on average a plurality of pores in walls separating adjacent cavities.

47. The particle of claim 39, wherein the average interconnecting pore diameter is at least 0.5 microns.

48. The particle of claim 39, wherein the average interconnecting pore diameter is 20% or less than the average cavity diameter.

49. The particle of claim 39, wherein the particle is formed from a mixture of monofunctional monomers.

50. The particle of claim 39, wherein the particle is formed from a mixture of polyfunctional crosslinking agents.

51. The particle of claim 39, wherein the cavity size is in the range of about 1 to about 50 microns in diameter.

52. A modified particle according to claim 39, wherein:
a. the particle is functionalized;
b. the particle is carbonized;
c. the particle has a gel or pre-gel deposited within the particle cavities; and/or
d. the particle has a chemical, pharmaceutical, cosmetic, formulation or combination thereof deposited within the particle cavities

53. A combination of different particles or groups of particles according to claim 39, wherein the different particles or groups of particles differ in shape, size, color, and/or included material.

54. The combination according to claim 53, wherein the different particles or groups of particles differ in color, and said color indicated the identity of a composition within that particle or group of particles.

55. Use of the particle of claim 39:
a. as a substrate in separation technologies;
b. in solid phase synthesis;
c. as a substrate for immobilizing a molecule such as a polypeptide, an enzyme, an oligonucleotide or other macromolecule;
d. in cell culture methods;
e. to contain whole viruses;
f. as carriers of active ingredients such as pharmaceutical agents;
g. as carriers for various cosmetic formulations and skin care applications;
h. to contain a catalyst; or
i. as an adhesive carrier.

56. A process for producing a porous, crosslinked polymeric particle comprising:
(a) combining to form an emulsion
(i) an oil phase comprising
(1) an oil-soluble, monofunctional monomer;
(2) an oil-soluble, polyfunctional crosslinking agent;
(3) an emulsifier that is suitable for forming a stable water-in-oil emulsion; and
(ii) an aqueous discontinuous phase,
wherein the emulsion comprises at least about 70% aqueous discontinuous phase;
(b) adding the emulsion to a mold having a particle volume of about 65 mm³ to about 525 mm³; and
(c) polymerizing the emulsion.

57. The process of claim 56, wherein the oil phase additionally comprises a porogen.

58. The process of claim 56, wherein residual components are washed from the particle without grinding the particle.
